(19) United States
(12) Patent Application Publication
(10) Pub. No.: US 2004/0082032 A1
(43) Pub. Date: Apr. 29, 2004

(54) Cctra gene as a tool to produce male-only progeny in the mediterranean fruitfly ceratitis capitata

(76) Inventors: Pasquale Delli Bovi, Portici (IT); Attilio Pane, Massa Lubrense (IT); Catello Polito, Gragnano (IT); Giuseppe Saccone, Napoli (IT)

Correspondence Address: CLARK & ELBING LLP, 101 FEDERAL STREET, BOSTON, MA, 02110, US

(21) Appl. No.: 10/471,211

(22) PCT Filed: Mar. 8, 2002
(86) PCT No.: PCT/IB02/00693

(30) Foreign Application Priority Data
Mar. 8, 2001 (IT) RM2001A000120

(51) Int. Cl. C07K 14/72; C07H 21/04; C12N 5/06

(52) U.S. Cl. 435/69.1; 435/320.1; 435/348; 530/350; 536/23.5

(57) ABSTRACT

This invention refers to the identification of the Cctra gene (SEQ.ID.NO. 1) and to corresponding dsRNA molecules comprising Cctra gene.sequences as a tool to produce only-male progeny in the Mediterranean fruitfly Ceratitis capitata.

### FIELD OF THE INVENTION

The present invention concerns the Cctra gene as tool to produce male only progeny in the Mediterranean fruitfly Ceratitis capitata.

### PRIOR ART

Ceratitis capitata, known as Mediterranean fruitfly (medfly) is a well known dipteran species because of the damage caused to agriculture in Italy and in other Mediterranean regions as well as in North, Central and South America (Robinson and Hooper, 1989). The Medfly, from Occidental Africa, has invaded wide regions, migrating in the last century, probably because of the intensification of transports and of agricultural cultivations, not only in Europe but also in Australia and in America. Every female injects by an ovopositor hundreds of embryos, future larvae, in the fruitcrops of more then 200 vegetal species, 100 of which are of economical importance (Christenson and Foote, 1960). Furthermore its presence in a country causes severe restrictions for the export of agricultural products because of the necessary quarantines imposed by the national and international organizations, such as for example the APHIS (Animal and Plant Health Inspection Service) in USA (Malavasi et al., 1994). For example, USA, New Zealand, Cile and Japan, which presently do not have medfly infestations, prohibit the import of fresh fruits from countries which medfly has not been eradicated. The damage is caused by the larvae which live and feed into the fruit pulp of the plant host and by the sting of the ovopositor which let the microorganisms to enter into the fruit causing its decay and its premature falling (Mitchel and Saul, 1990). In Central America, excluding Mexico and Panama, it has been estimated that medfly has caused annual losses of more then 25 millions of US dollars and in Mexico about 800 millions of US dollars (Gibbs and Eerde, 1981). The sterile insect technique (SIT) is used as eradication strategy for the medfly, in alternative to pesticides (the more used in USA is malathion), or, in some cases, in combination with their reduced quantities (California, USA). The SIT is a method of control that does not pollute the environment and that is based on the massive rearing, the sterilization and the release of a huge number of flies (Knipling, 1955). Furthermore, differently to the pesticides, it is species-specific and do not eliminate beneficial species, as the impollinators, or natural species-enemies. The mating of the sterile released males with the native females determines a reduction of the reproductive potential of the infestating population and its eradication when a sufficient quantity of males are released for a long period of time. The success in SIT application in little scale experiments against medfly in Hawaii, Costa Rica, Italy and other regions in the world, has led to apply it on massive scale in Mexico, Peru, Guatemala, Chile, Argentina, Florida e California (Boller, 1987). The key event, that gives rise to success of the SIT, is the transfer of irradiated sperms (with X or Gamma rays) carrying lethal dominant mutations from the released males to wild type females. Considering that in Ceratitis, like in most insects, the sex ratio is 1:1, the SIT consists in releasing flies of both sexes. Nevertheless the reared females, irradiated and released together with males, do not contribute to the suppression of the infesting population: in fact males are polygamous and, after the sterile flies is releasing, will mate many times with the available females. Polygamy will increase the mating probability between the wild type males and the wild type females, but not with the sterile females, limiting the suppression effect that sterile females could exert. Instead, the sterile males will mate with several wild type females and they will determine a strong suppression effect on the resident population. Moreover the released females contribute to damage caused by the pest population perforating the crops that hence can deteriorate. Females could have a different sensibility to irradiations and so could be only partially sterilized with respect of irradiated males. Irradiated males, if released together with sterile females, are incline to mate with them rather then with wild type females. To potentiate the SIT, hence, have been developed Ceratitis sexing strain to select the male progeny, separating it from the female one, to sterilize it and to release it in the infested areas. The global control exerted by this strategy on the infesting flies is more efficient then the one obtained by releasing sterile flies of both sexes and furthermore the costs of irradiations and release are decreased with the same number of flies (Rendon et al., 2000). The prevention programs can be carried out without the fear to damage the agricultural products because of the stings caused by the released sterile females. Sexing strains have been developed by genetic methods based on the use of chromosomal translocations which let to link to the Y chromosome, and hence to the masculinity, specific selectable genes. By X or gamma rays irradiation at pupal or adult stages chromosomal breaks and traslocations have been induced into cellular nuclei of Ceratitis. After suitable crosses there are been selected lines bearing reciprocal Y-A (Y and Autosomal chromosomes) chromosomal translocations, identifying them on the basis of the reduced fertility of the males or using the pseudolinkage of Y-linked genetic markers (Fisher, 2000). By this way, there have been developed two types of GSS (Genetic Sexing Strains) strains: the first is based on the use of a mutation which causes a white color of the pupae, instead of the brown wild type color; the second, more recent and efficient, is based on a temperature sensitive embryonal lethal mutation (TSL). In both cases the wild type allele is present in a autosomal fragment translocated on the Y chromosome and the reciprocal Y fragment is, on the contrary, translocated on the autosome. The homologous autosome, not interested by the translocation, bears a mutant allele. These males are mated with females bearing the mutant allele in homozyogpsity. In this way the first translocated strain the male progeny is constituted by dark pupae, while the female one by white pupae. In the second strain, on the contrary, the XY embryos are able to survive an heat shock at 34° C. for 24-48 hours because they have a wild type allele of the tsl locus linked to the Y chromosome (as well as a Y fragment, not bearing the M factor, reciprocally translocated on the autosome), while the females bear the mutant autosomal allele in homozygosity (Fisher, 2000). It has to be seriously considered anyway that the use of TSL mutations and of chromosomal translocations determines a reduction of fertility in the mass reared strain and a periodic instability (chromosomal) of the sexing system. For this reason, in parallel to the classical genetics strategies in the last decades have been performed molecular genetics research activities to obtain new systems of male-only production in Ceratitis capitata (Louis et al., 1987; Furia et al., 1992; Saccone et al., 1996; idem, 2000).

The qualifications of an effective method for a male-only production of Ceratitis capitata flies, as well as for any other pest insect, are that the mutation would be stable during massive rearing conditions and that could avoid the lost of half reared population (the females). The authors attempted the strategy to induce in Ceratitis a complete sexual reversion of female flies into XX males and they are engaged in research by years with this aim to identify genes regulating sex determination of Ceratitis. For their aims, they can use a gene transfer technique in Ceratitis (Loukeris et al., 1995; Zwiebel et al., 1995) which let to stably insert into its genome exogenous DNA fragments. By this way, the authors propose to produce genetically modified strains bearing a transgene able to induce either a female-specific conditional lethality or, even better, a sexual transformation of females into males. As the Ceratitis sexing strains based on translocation and mutations are unstable under massive rearing conditions, the genetic manipulation based on the use of transposable elements could result into a preferable alternative solution with the respect of those available. Indeed it has been demonstrated that in Drosophila with a P transposable element and in Ceratitis with Minos transposone, that the insertions are stable during generations. The gene transfer technique using the Minos transposone is described in U.S. Pat. No. 05,348,874. In the genetic model D. Melanogaster the sex of the individuals is determined by the number of X chromosome with respect of the number of autosomal aploid set (X:A ratio; XX:AA32 1 females; XY:AA=0.5 males). The X:A primary signal acts as the only key gene, Sex-lethal (Sxl) activating it exclusively in XX):AA embryos (Cline and Meyer, 1996; Keyes et-al., 1992). Sxl, then, activates during larval stages only in the females the transformer (tra) gene and this modifies the expression of the doublesex gene (dsx), so that the male sexual differentiation is repressed and the female one promoted. In XY embryos the absence of Sxl activation and hence of tra, determines a different activation of dsx, which on the opposite, promotes a male differentiation, repressing the female one (Burtis and Baker, 1989). The sex determination is based on a regulatory cascade in which the sex-specific alternative splicing produces differently spliced transcripts starting from Sex-lethal (Sxl), transformer (tra), transformer-2 (tra-2) and doublesex (dsx). In Drosophila it has been possible to isolate strains mutant in these genes (or to produce transgenic strains) in which a specific cross or an heat shock are sufficient to reproduce unisexual progeny (McKeown et al., 1988; Polito et al., 1990; Fortier and Belote, 2000). Hence Drosophila is a reference to study control strategies of those insects dangerous for agriculture (Ceratitis capitata) or for human health (Anopheles gambiae, known malaria carrier). Although the key position played by Sxl gene in this regulatory cascade, homologous Sxl genes, isolated in dipteran species belonging to families other then Drosophilidae, such as Ceratitis capitata (Tephritidae), Musca domestica (Muscidae) and Megaselia scalaris (Phoridae; more then 200 millions years from Drosophila) do not present a conserved sex-specific regulation (Saccone et al., 1996; Sievert et al., 1997; Meise et al., 1998; Saccone et al., 1998; Schutt and Nothiger, 2000). These observations suggest that the role played by Sxl in the sex determination is not evolutionarily conserved in insects. Also the primary signal for sex determination of Ceratitis is different in Ceratitis capitata with the respect of Drosophila. Indeed, it consists of one or more male-determining factors, whose molecular nature is presently unknown, localized in proximity of the centromere on the long arm of the Y chromosome (Willhoeft and Franz, 1996). Hence, it is presently unclear if within the insects the sex determination is an evolutionarily conserved genetic mechanism.

### SUMMARY OF THE INVENTION

The present invention is base on the hypothesis that the tra gene is evolutionarily conserved. This hypothesis has been verified by the authors isolating the Drosophila tra homologue in Ceratitis (Cctra) and revealing that it is sex-specifically regulated by alternative splicing. Hence, it is proposed that in the dipteran species in general and in Ceratitis in particular the regulatory segment tra>dsx of the Drosophila cascade is evolutionarily conserved. To investigate the in vivo function played by the Cctra gene we have used the gene silencing technique (also called RNA interference) by dsRNA molecules (ds, double strand RNA) containing Cctra specific sequences. Injecting dsRNA molecules, artificially produced using a Cctra cDNA as template for in vitro transcription, we demonstrate that it is possible to induce a sexual reversion of the phenotype from female to male one. This experiment show also that the Drosophila tra gene is functionally conserved in a dipteran species belonging to a family (Tephritidae) moderately distant from a phylogenetic point of view from Drosophililiae (120 M. A; Beverley and Wilson, 1984). The object of our invention consists hence in the identification and isolation of a gene, Cctra, of Ceratitis which functions as main sex determination regulator and into a ad hoc genetic manipulation of this gene to use it in gene silencing experiments, a technique previously developed by other authors (Fire et al. 1998, Kennerdel and Carthew, 1998). Indeed we demonstrate, as described in the following sections, that injecting dsRNA molecules complementary to the Cctra sequence during early embryonal stages it is possible to transform the sexual phenotype of Ceratitis masculinizing in a apparently complete way flies having an XX caryotype (females).

Another object of the invention is the production of male-only progeny in dipters in general, particularly those harmful or detrimental to human health such as Glossina (sleeping sickness fly), Anopheles gambiensis (malaria vector) Aedes Aegypti (dengue vector), more particularly in the Mediterranean fruitfly Ceratitis capitata.

Other objects of the invention concern the plasmids and vectors produced to execute the method of our invention, concern also the cells including these vectors and the transgenic animals non human including theses cells.

Further objects will be evident from the detailed description of the invention.

### BRIEF DESCRIPTION OF THE FIGURES

FIG. 1 Analysis by Northern blot of the Cctra transcripts produced by the Cctra gene in adult males and females. Two male-specific transcripts of 1.9 Kb and 1.7 Kb and two female-specific transcripts of 3.5 and 1.6 Kb are detected.

FIG. 2 Nucleotide sequence Seq. IDN. 1 of the cDNA F1 derived from the female-specific transcript of the gene Cctra. It is reported also the aminoacid Seq. IDN 2 deduced corresponding to the protein CcTRAF long 429 aa. The symbol with a V shape indicates the positions of the intron-eson sides. The two arrow indicate the positions of the two oligonucleotides used to produce the dsRNA molecules.

FIG. 3 Alignment of the putative aminoacid sequences of the various homologous TRA proteins of Drosophilidae and of C. capitata. The regions more conserved are indicated with the 4 box numbered in which there are marked in bold the aminoacid conserved in all the aligned proteins. In blue are indicated the aminoacid positions common to CcTRA, DvTRA e DhTRA. Furthermore the vertical arrows indicate the positions of the introns in the corresponding genes.

FIG. 4A Analysis by RT-PCR of the transcripts produced by the gene Cctra in the females (2) and male (4) adults of Ceratitis. In particular it is shown a scheme which indicate the positions of the two oligonucleotides used in the reactions of the amplification with the respect of the various exons of which is composed the locus Cctra.

FIG. 4B Analysis by RT-PCR of the transcripts produced by the gene Cctra in the females (2) and male (4) adults of Ceratitis. In particular it is shown the result of an electrophoretic run of the RT-PCR reactions. The cDNA fragment amplified in females is about 1.3 Kb long, as expected on the basis of the F1 clone sequence (see FIG. 2); the cDNA fragment amplified in males results longer and about 1.6 Kb long. This male-specific product has been cloned and partially sequenced (data not shown). Its sequence shows the presence of two male-specific additional exons reported in A with the blue color.

FIG. 5A There are shown respectively below a wild type female (Benakeion) and above an injected fly (Benakeion)

FIG. 5B It is shown a male with a normal sexual phenotype (strain white eye)

FIG. 5C It is shown a male without antennae (strain white eye, posterior pole injections)

FIG. 5D There are depicted above a female (white eye strain) and below an injected female which it has an anomalous external reproductive apparatus.

FIG. 5E External reproductive apparatus of the female of FIG. 5D. indeed it is possible to observe this region magnified which it has an aspect FIG. 5F Female of FIG. 5D deprived of the typical ovopositor.

FIG. 5G The Ceratitis female does not show into the eye a blue reflex

FIG. 5H The Ceratitis male shows into the eye a blue reflex

FIG. 5I The blue reflex present in the intersexes injected at the posterior pole that show a masculinized head and a female reproductive apparatus.

FIG. 6 Lanes of electrophoretic run of genomic DNA amplifications from males and females of the white eye strain.

FIG. 6B It is shown a control experiment to ascertain the presence of genomic DNA in every sample of those analyzed in FIG. 6A.

### DETAILED DESCRIPTION OF THE INVENTION

In the description the words cDNA F1 and cDNA M1 are referred respectively to the female-specific and male-specific cDNAs. Functional results obtained from the authors can be reproduced also with the nucleotide and aminoacid sequences having an identity of 40% or more, preferable of the 60% or more, more preferable of the 80% or more preferable of the 90% or more to the sequence here reported in the following sections (Yang et al., 2000). Given the unusually low degree of sequence conservation among tra homologues in Drosophila species (O'Neil and Belote, 1992), we decided to attempt the isolation of the tra gene in Ceratitis avoiding the low stringency hybridizations and exploiting the gene I(3)73Ah (sequence AE003526[ACCN]). This gene is physically linked to the tra gene (the 3′ untranslated regions of the two genes partially overlap), this linkage is conserved in other Drosophilidae species and the gene is highly conserved also in vertebrates (O'Neil and Belote, 1992; Irminger-Finger and Nothiger, 1995).

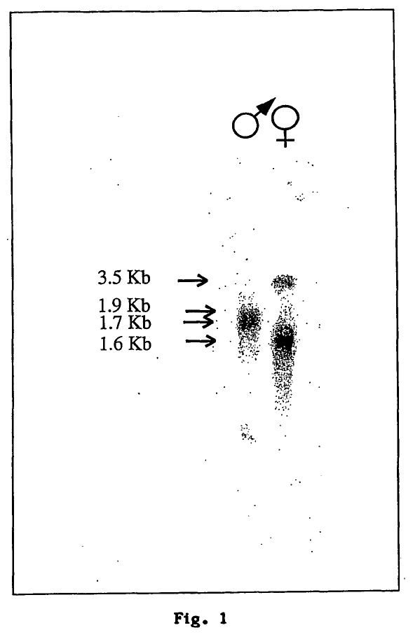

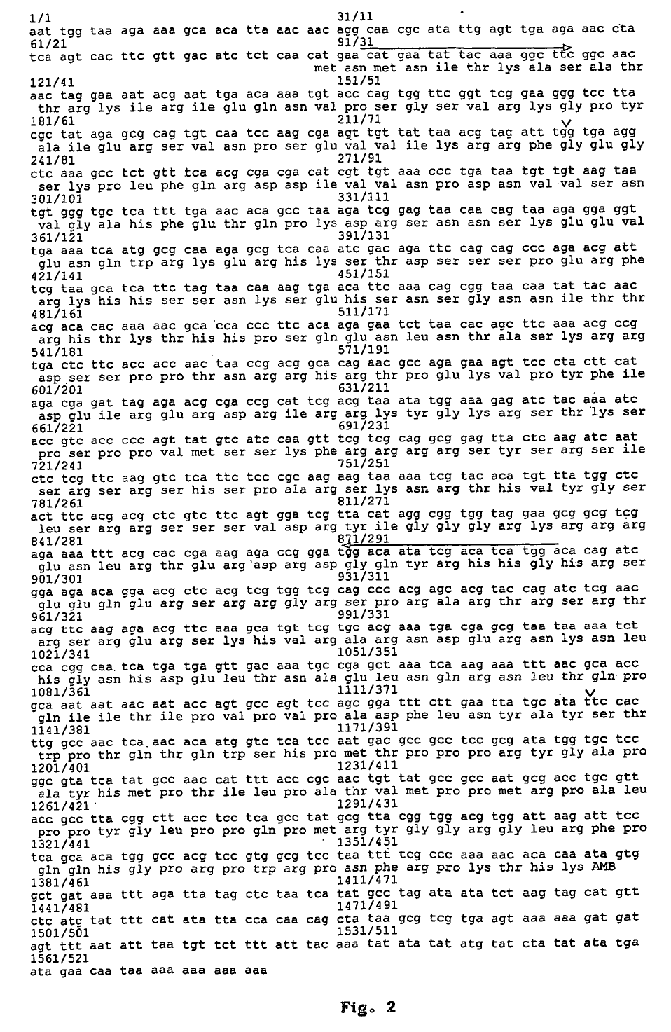

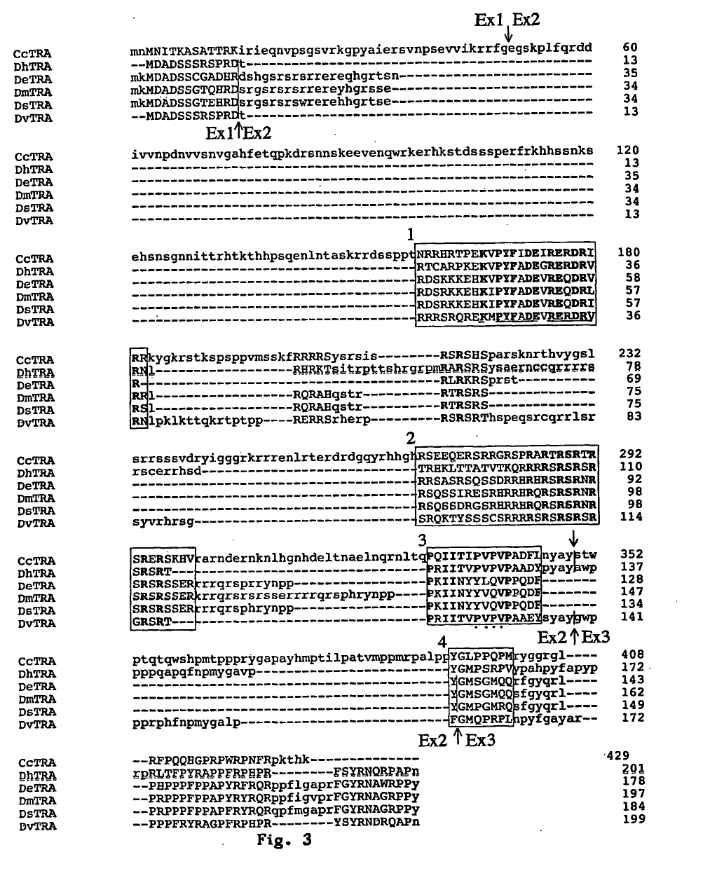

We hence isolated a region from the Ceratitis genome containing the I(3)Ah homologue and within its proximity the transformer homologue, which we named Cctra. The gene Cctra produces, as in Drosphila, sex-specific transcripts of different length. Indeed a clone of cDNA (F1) subsequently isolated from a cDNA library of adult females and used as radioactive probe in Northern blot experiments detects in females two mRNAs of about 3.5 Kb and 1.6 Kb and in males two mRNAs of about 1.7 Kb and 1.9 Kb (FIG. 1). The cDNA F1 contains an insert of 1.6 Kb and it could corresponds, hence, to the female-specific transcript of similar length. The F1 clone analyzed by sequence presents an Open Reading Frame (ORF) which encodes a 429 aminoacid long protein, rich in serine and arginine (which constitutes the so called SR motif) and with short regions of similarities with the TRA proteins of Drosophilidae (FIG. 2 and 3).

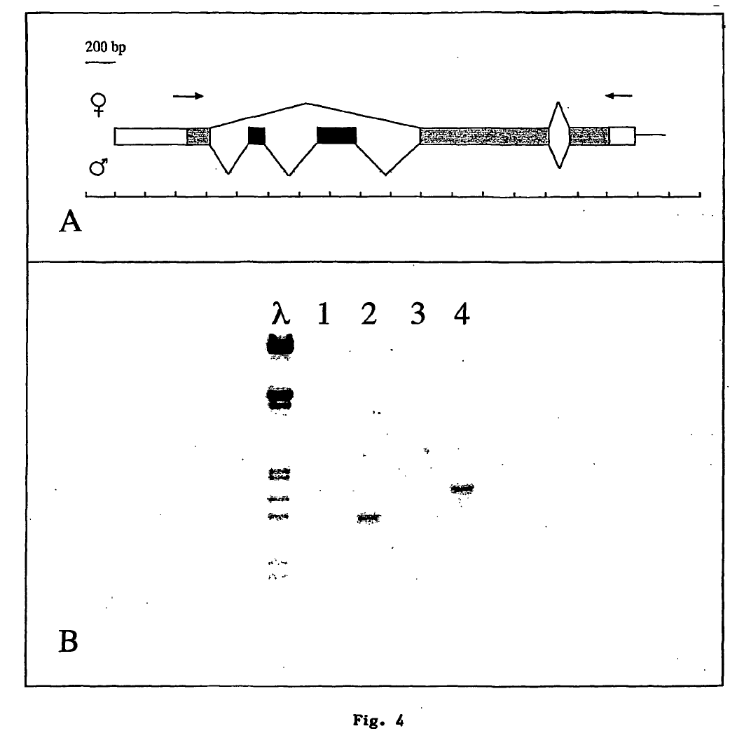

In an RT-PCR experiment two oligonucleotide (F+ and Z1−) have been amplified from the RNA extracted from females and males, two products respectively of about 1.6 Kb and 1.3 Kb (FIG. 4). The female-specific product of 1.3 Kb has a length similar to the one expected on the basis of the F1 sequence. The product of 1.6 Kb, which on the contrary seems to be longer, has been cloned into a plasmid (clone M1) and it has been analyzed by DNA sequencing. Comparing the sequence of the M1 clone with the one of the F1 clone it has been possible understand that it contains two additional exons, inserted into the coding region and respectively 40 and 197 nt long. The first of these two male-specific exons introduces a stop-codon which determines a possible translation of a CcTRA protein of only 59 aminoacids.

The partial determination of the nucleotide sequence of a genomic clone and the comparison with the sequences of the F1 and M1 clones has led to understand that the Cctra locus is composed of 5 exons, 2 of which are male-specific (FIG. 4A).

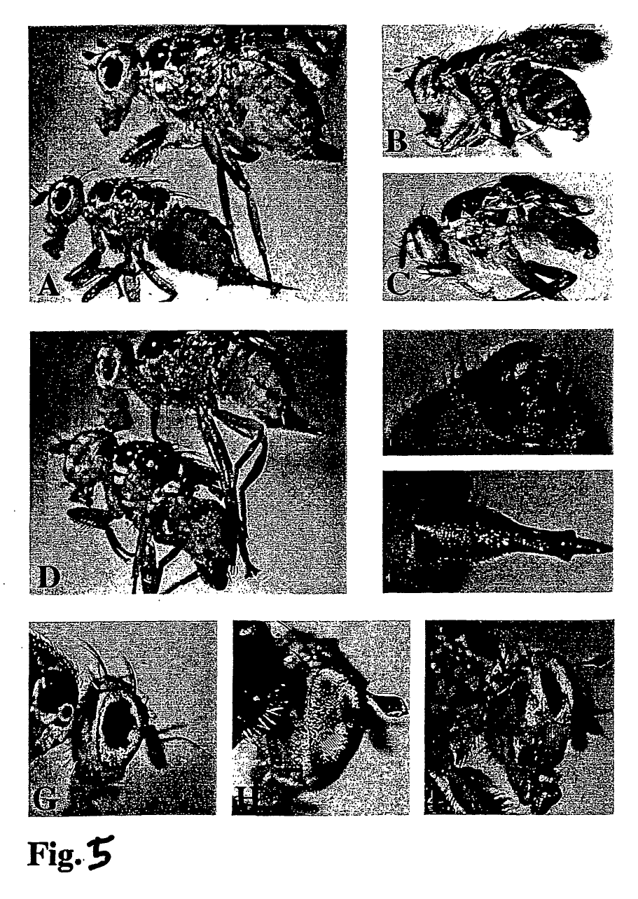

The functional analysis of the transformer gene of Ceratitis capitata has been performed by in vivo experiments based on the “RNA interference” technique (RNAi) (Fire et al., 1998). For this aim dsRNA molecules corresponding to exonic regions of the Cctra gene have been injected into the Ceratitis capitata embryos. In particular the chosen region for the RNAi is extended from the position 154 (exon 1) to the position 893 (exon 5) of the female-specific Cctra F1 cDNA (FIG. 5) and corresponds to exonic regions present into the cDNA F1 and M1 clones.

To produce dsRNA of Cctra has been designed 4 syntetic oligonucleotides named: 154+, 154+/T7, 893− and 893−/T7 on the basis of the F1 Cctra cDNA sequence. The oligonucleotides 154+/T7 and 893−/T7 bear at the 5′ end the sequence of the T7 phage RNA Polymerase promoter. Using these oligonucleotides into the combination 154+ with 983−/T7 and 164+/T7 with 893− and the cDNA Cctra F1 as template, have been amplified by PCR DNA fragments of about 0.8 Kb which differs only for the position of the artificial promoter.

Subsequently, starting from these fragments it has been performed an in vitro transcription which permitted to obtain two single stranded sense and antisense RNA (ssRNA). Then equimolar quantities of the two ssRNA have been mixed into a solution of annealing buffer to produce double stranded RNA and the dsRNA has been transferred into the injection buffer. 300 embryos of the white eye Ceratitis strain have been injected with the produced dsRNA, into the posterior pole, and 210 embryos of the Benakeion strain into the anterior pole. From the injected embryos 149 developed from the white eye strain (embryonal survival rate of 49.6%) and 89 larvae of the Benakeion strain (embryonal survival rate 42.3%) (Tab. 1). There have been subsequently collected 84 pupae white eye (larval survival rate of 56.3%) and 68 pupae Benakeion (larval survival of 76.4%). Then 69 flies white eye developed (total survival rate of adults with the respect of embryos 23%) and 48 Benakeion flies (general survival rate 22.8%). The adult flies can be classified in different phenotypic classes as reported in the Tab. 1.

TABLE 1

| 69 white eye flies from | 48 Benakelon flies from |
|---|---|
| the embryos injected at | the embryos injected at |
| the posterior pole | the anterior pole |
| 61 flies with a male phenotype | 29 flies with a male phenotype |
| 6 flies with a male phenotype | 10 flies with a female phenotype |
| lacking of antennae | but with antennae |
| 1 fly with a female phenotype and | 6 flies with a male phenotype |
| with fused genitals | without genitals |
| 1 fly with a female phenotype | 3 flies with a female phenotype |

The ratio between the sexes of the born flies from the two experiments is clearly unbalanced in favor of the male sex. In the flies born from the injection into the posterior pole of the white eye embryos the percent of the males versus the females is 98.3%, while for the flies born from the injections into the anterior pole of the wild type strain Benakeion, the percent of males versus females is 90.6%. These percents could be due to female-specific letality or to a complete masculinization of the XX female flies (the percents have been calculated considering only those flies with an apparently normal phenotype). Furthermore, are present flies with intermediate phenotypes, which we can define as intersexes. These intersexes show, a partial sexual transformation, localized to the region proximal to the injected pole. Indeed from the injections at the posterior pole of the embryos of the white eye strain have been obtained 6 intersexual flies with a male reproductive apparatus, but with a female-like head (eyes with green reflexes and absence of “antennae”). From the injections at the anterior pole of the wild type Benakeion strain, on the contrary, there have been developed 10 intersexual flies with a female reproductive apparatus, but with a male-like head (presence of “antennae” and blue eye reflexes). There have been obtained also 7 flies out of 117 (1 female and 6 males), with malformations of the external reproductive apparatus, which is only sketched or almost completely absent.

In FIG. 5 there are reported the different phenotypic classes obtained. In the FIG. 5A, there are shown respectively below a wild type female (Benakeion) and above an injected fly (Benakeion), that shows a mosaic intersexual phenotype, with the female abdomen and the male head (see the male-specific antennae). In 5B and 5C have been shown respectively a male with a normal sexual phenotype (strain white eye) and one without antennae (strain white eye, posterior pole injections), a phenotypic trait explainable as incomplete mascunilization of the head. In FIG. 5D are depicted above a female (white eye strain) and below an injected female which it has an anomalous external reproductive apparatus. In FIG. 5E indeed it is possible to observe this region magnified which it has an aspect clearly different from the normal wild type female form (FIG. 5F) and it is deprived of the typical ovopositor.

The Ceratitis male shows into the eye a blue reflex (FIG. 5H) which is absent in the female (FIG. 5G) but which it is present in the intersexes injected at the posterior pole that show a masculinized head and a female reproductive apparatus (FIGS. 5I and 5A above).

In the few intersexual flies the correlation between the injection site (anterior-posterior) into the embryos and the local effect on the sexual phenotype of the adult (head-abdomen) it is explainable with the fact that the development of the dipteran species is based on the system of anterior-posterior and dorso-ventral coordinates, in part already active into the egg and then into the embryos which establish during these early stages “which” are the various cellular groups that will give rise to the various adult structure (Carroll, 1998).

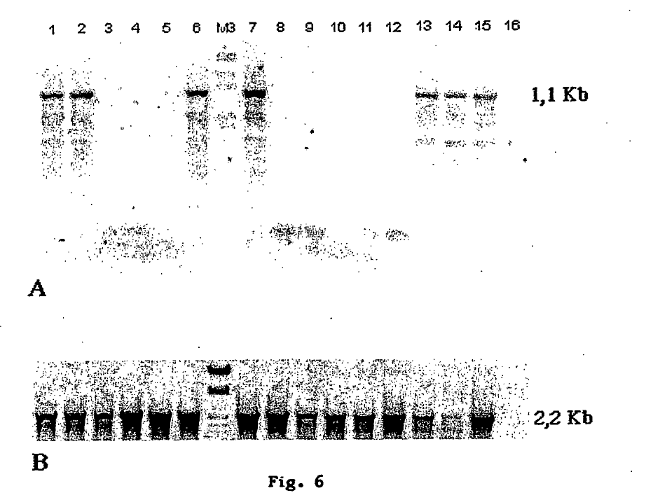

These data obtained strongly suggest that the injection of dsRNA specific interferes with the normal expression of the Cctra gene, repressing, as expected, its normal function into the injected individuals. The difference observed between the number of the flies of the two sexes could be explained with two different hypothesis: 1) the “silencing” of “transformer” is lethal to the females which then cannot develop into adults; 2) the females injected with the dsRNA develop following a complete sexual reversion into males apparently normal but with XX karyotype. To distinguish between the two hypothesis we determined the karyotype of 10 flies of males phenotype born from the injection at the posterior pole of embryos of the white eye strain. Genomic DNA extracted from a single fly has been the subject of PCR experiments based on the use of oligonucleotides which amplify specifically a region of the Y chromosome of about 1 Kb (Anleitner and Haymer, 1991). As positive control of these experiment have been included into the PCR also genomic DNA extracted from males and females of the white eye strain. The results of this experiment are shown in FIG. 6A. In the lanes of an electrophoretic run from 1 to 10 the DNA amplified from the injected flies of the white eye strain are shown. In the lanes 11 and 12 of the electrophoretic run it is visible the amplified from the genomic DNA extracted from white eye females. In the lanes 13 and 14 it is reported the result of the PCR on white eye males. In the lane 16 it is present a negative control. It is possible to see that only 4 flies (1-2-6-7) have an amplification pattern characteristic of the white eye males (Y-specific fragment of 1.1 Kb; the other bands are due to the amplification of other copies of this repetitive element) of the white eye males (11-12 two white eye males not injected), while the remaining 6 show (34-5-8-9-10) a female-specific pattern comparable to the one in 13-14 (two white eye females not injected). Into the electrophoretic run 15 it is observed the amplified of the genomic DNA extracted from white eye female and males flies. In FIG. 6B it is shown a control experiment to ascertain the presence of genomic DNA in every sample of those analyzed in FIG. 6A. In this case there have been used as primers two synthetic oligonucleotides, Cctra 154+ and Cctra 1113− which amplify a genomic fragment of about 2.2 Kb from the Cctra gene. Hence it is possible to conclude that only 4 out of 10 males examined have an XY karyotype, while the remaining have an XX karyotype. These ones have to be considered females reverted into males apparently normal.

To verify if the XX males are fertile, we individually crossed the males obtained from the injected embryos with wild type females. Some crosses gave progenies of both sexes, while other crosses gave female only progeny, suggesting that the XX males are able to produce functional sperms containing the X chromosome and able to successfully mate with the wild type females. Analysis by PCR led to demonstrate that these fertile males had XX cariotype. These data indicate, hence, that the sex distortion in favor of the males observed into the Tab 1 is explainable with a complete sexual reversion of XX females instead of a female-specific letality.

As in Drosophila the transformer gene of Ceratitis capitata (Cctra) is strictly associated and overlapped to the I(3)Ah gene, encodes a serine-arginine rich protein, produces transcripts of different length in the two sexes by alternative splicing (McKeown et al., 1987) and it is necessary for the normal development of female flies, while it is dispensable for male development (Sturtevant, 1945). We have demonstrated that RNAi interference technique (RNAi) is successfully appliable to Ceratitis capitata and that RNAi allows the production of male only progeny when Cctra sequences are employed. Indeed microinjection of double-stranded RNA (dsRNA) corresponding to portions of Cctra exonic regions, in early embryos leads to a sexual transformation of females into XX males. XX males are phenotypically indistinguishable from wild type XY males. On the contrary XY embryos are unaffected by RNAi and develop into apparently normal XY males. The sexual reversion of females into XX males is complete, as demonstrated by the fertility of these males. This experimental data is important because it strongly suggests that the XX males, as the XY, once sterilized are able to compete with the wild type males for the mating and that hence the XX males as the XY are able to contribute to the reduction of the progeny in the next generation. Our technique, with the respect of the TSL Ceratitis strain already available, offers hence the possibility to avoid the lost of the 50% of the reared population, comprising the females, transforming it into a male population useful to control the infesting population. With similar mass rearing costs, it could be hence produced a double number of sterile males.

Two classes of intersexes where also obtained, which display both male and female characteristics, namely females with antennae and males without antennae. These flies are probably the result of an incomplete diffusion of the dsRNA molecules through the injected embryos. Therefore a strict correlation between the injected embryonal pole and the adult sex-reversed body part is clearly detectable. Among RNAi individuals a few unaffected XX females were observed, which have probably developed from sporadic untreated embryos (for example, the embryos is perforated but it has not been properly injected with the solution). On the basis of our findings we propose to use of the nucleotide sequence of the cDNA clone named CctraF1, that contains exonic regions of the Cctra gene (FIG. 2), to eliminate from the progeny the presence of the females, transforming them into males of Ceratitis whenever it is used to sex Ceratitis flies throughout the technique we employed. We have shown a functional conservation of the Drosophila transformer gene, as main regulator of the sex determination also in Ceratitis, we propose that our invention will be extended to many other insects species dangerous for the agriculture, for the human health and the health of farm animals. Utilization of dsRNA corresponding to Cctra sequences other than CctraF1 will interfere with Cctra function and promote sex reversion events of Ceratitis.

Gene silencing mediated by RNAi can be achieved by generating transgenic strains (Fortier and Belote, 2000) of Ceratitis which contain inducible transgenes expressing dsRNA molecules specific for Cctra.

With this method the interference is believed to be maximal and the progeny will be constituted exclusively of XY males and XX flies completely masculinized. Other molecular manipulations on this gene and/or its products could give similar results. It is conceivable the other techniques-interfering with Cctra function can produce results similar to that of the RNAi. For instance microinjection of antisense DNA and/or RNA molecules or of CcTRA antibodies (antibody blocking) or antimorphic TRA proteins could induce a “knock-out” of Cctra function and a masculinization of the XX flies. However these techniques are supposed to be less efficient than Cctra dsRNA molecules microinjecion. To permit a large scale application of our methods and the mass-rearing of Ceratitis male-only populations, we have planned the following alternative or complementary projects: 1) production of transgenic strains able to produce Cctra-specific dsRNA (Fortier and Belote, 2000; Kennerdell and Carthew, 2000; Lam and Thummel, 2000); 2) development of a method to introduce dsRNA molecules into early embryos alternative to single embryo micro-injections as for instance electroporation or lipofection; 3) development of transgenic strains expressing CcTRA-GFP fusion proteins in embryos in order to automatically separate XX (displaying a specific color) from XY (displaying a different color) embryos throughout cell-sorting like apparatus (Sorensen et al. 1999); 4) substitution of the endogenous Cctra gene with a modified Cctra transgene via homologous recombination to transform females into males (Rong and Golic, 2000). In particular the point 3 proposes our original idea to apply to Ceratitis embryos a sorting technique developed for the cells and based on a different color in vivo that cells and organisms can show if they express a transgene encoding a Green Flourescent Protein (GFP). The first molecular cloning of a transformer homologue in a non Drosophilidae species like Ceratitis capitata led to demonstrate a structural, regulatory and functional conservation of the gene and to the possibility of identifying homologues in other species. Considering that Drosophila and Ceratitis belong to different dipteran families, that in the group of Acalyptratae (including about 60 of the 120 families) are among the more distantly related from a phylogenetic point of view (120 millions of years), we propose that the transformer gene is a key regulator of sex determination in many other dipteran species also of economical and medical-sanitary importance. Aminoacid and nucleotide alignments among Tra proteins and tra genes of Drosophila species and Ceratitis allowed us to identify short regions with significant sequence identity (see FIG. 3). Such sequences were used to produce degenerate oligonucleotides to search for tra homologues in economically (as a number of Bactrocera species) and sanitary (as Glossina, Anopheles, etc.) relevant species.

Many species belonging to the Tephritidae and hence closely related to Ceratitis represent agricultural pests: for instance Bactrocera oleae, also known as Dacus, is a great problem for olive cultures in the Mediterranean area; Bactrocera tryoni, Bactrocera musae (banana fruitfly) and Bactrocera papayae (papaya fruitfly) are well known in Australia. The cloning of tra homologues in these species will open the possibility to develop control strategies analogous to that we are currently setting for Ceratitis which are based on the Sterile Insect Technique.

The following examples are given useful to better show the invention and they are not to be considered limitative of its scope.

### EXAMPLES

All standard techniques of molecular biology to perform blotting, ibridization, enzimatic restriction, chimeric plasmids were essentially those described by Maniatis et al. (1982).

### Example 1

Genomic DNA Extraction From Single Fly

Genomic DNA extraction was carried on as described by Andrew and Thummel (1994) adapting the protocols to a single Ceratitis fly. Single flies were pottered with a pestel in eppendorf containing 200 μl lysis buffer (20 mM Tris, pH 7.5, 0.2 M NaCl, 20 mM EDTA, 2% SDS). 20 μl of 250 μg/ml Proteinase K (BOEHRINGER MANNHEIM) were added and the solution was then incubated at 50° C. for 1 hour. After the incubation step 100 μl of Phenol were added and the solution was vortexed for 5′ minutes. 100 μl Chloroform were added, the mix was vortexed for 5′ minutes and centrifuged at 13000 rpm for 5 minutes. The water phase was transferred to a clean tube containing 200 μl Chloroform. After 5 minutes vortexing the solution was centrifuged at 13000 rpm for 5 minutes and the aqueous phase was transferred to a clean tube. 400 μl 96% Ethanol were added and the tube was incubated at −20° C. for 2 hours. Then the solution was centrifuged at 13000 rpm for 5 minutes at 4° C. Finally the pellet was resuspended in 30 μl TE 1×, 10 units of RNAse DNAse-free (PROMEGA) were added the tube incubated at 37° C. for 2 hours.

### Example 2

Caryotypic Analysis by PCR

To amplify Y-Specific sequences the Taq-DNA Polymerase (AMERSHAM PHARMACIA) and the Perkin Helmer Gene Amp 9600 apparatus. Primers used in the PCR experiments were the following:

1-Y-SPECIFIC: 5′ GCGTTTAAATATACAAATGTGTG 3′ (SEQ. ID. NO. 3)

1 Kb Y-SPECIFIC: 5′ TACGCTACGAATAACGAATTGG 3′ (SEQ. ID. NO. 4)

0.4 μgenomic DNA were used as template in each PCR reaction. The PCR program was made up of a denaturation step at 94° C. for 5′, then 35 cycles as follows: 94° C. for 1′, 60° C. for 1′ and 72° C. for 1′. PCR product were analyzed on agarose gel.

The primer used to amplify a DNA fragment from the Cctra locus has the following sequence:

Cctra 1113-5′ CTGGAACTGGCACTGGTATTG 3′ (SEQ. ID. NO. 5)

### Example 3

Northern Blot

Total RNA was prepared from embryo, larvae and adult of the Benakion strain using Guanidinium Isothiocyanate (SIGMA) and ultracentrifugation in CsCl gradients as described by Maniatis et al. (1982); poly(A+) enrichment was obtained throughout chromatography with oligodT columns (CLONTECH). We separated 4 μg polyA(+) RNA per each lane by 2.2 M formaldehyde gel electrophoresis and buffer RB and transferred RNA onto a Hybond NX membrane filter (Amersham). For hybridization, a Cctra probe was prepared by nick-translation labelling (GIBCO BRL) of a CctraF1 cDNA fragment (from position 99 to 1495) in the presence of [α³²P]dCTP (NEN). Autoradiographic analysis was performed with films (KODAK) or with Phosphorimager (MOLECULAR DYNAMICS).

### Example 4

RT-PCR Analyses of Transcripts

RT-PCR retrotranscription was made using the oligodT as primer and the SuperScriptll enzyme (Gibco BRL). RT-PCR was performed with the following primers:

F+=5′ CATGAACATGAATATTACAAAGGC 3′ (SEQ. ID. NO. 6)

Z1−=5′ CACGACGCTTATAGCTGTTGT 3′ (SEQ. ID. NO. 7)

These primers were derived from female-specific CctraF1 cDNA with 5′ end at positions 88 and 1484. Cycling conditions were denaturation at 94° C. for 5′, followed by 35 cycles of: 94° C. for 1′, 60° C. for 1′, 72° C. for 2′30″. The PCR products were cloned in pUC-18 vector (Sure Clone Ligation Kit, AMERSHAM-PHARMACIA-BIOTECH) and sequenced by T7 Sequencing Kit (BYOLABS). The sequence alignment have been performed by Macaw, Blast and FASTA software. To search data bases there have been used the following on line software: Blast (EBI), Blast (Japan), Flybase and Blast NCBI.

### Example 5

Library Screening

To isolate the genomic clone containing the transformer gene an EMBL3 genomic library of Ceratitis capitata has been used (from the Benakeion strain). The molecular probe used in this experiment corresponds to a region cDNA of the gene CcI(3) (Pane et al., unp. Res.) labeled by incorporation of (α³²P)dCTP. A screening has been performed on a Ceratitis adult females cDNA library (prepared with RNA extracted from the Benakeion strain; Saccone et al., 1998). The molecular probe corresponds to a fragment of the Cctra gene obtained by enzymatic digestion and labeled by (α³²P)dCTP (unp. res.). The hybridization has been performed at 65° C. for 14 h in a solution of 5× SSPE, 0.1% SDS, DENHARDT 5×, DNA carrier (LIFE-TECHNOLOGIES) 0.1 mg/ml. Subsequently the filters have been washed 3 times for 15′ at room temperature into a solution of SSPE 2×, SDS 0.1%; 1 wash at 37° C. for 30′ into a SSPE 2×, SDS 0.1%; 1 wash at 65° C. for 2 h into SSPE 2×, SDS 0.1%; 1 wash at 65° C. for 30′ into SSPE 2×, SDS 0.1%.

### Example 5

Preparation of the Cctra dsRNA Molecules and Injection into the Ceratitis capitata Embryos

The Cctra dsRNA has been produced by in vitro transcription using as DNA template PCR products. To obtain the DNA templates there have been used the following primers (the number indicate the positions on the clone F1 sequence):

Cctra 154+: 5′ CAGTGGTTCGGTTCGGAAG 3′ (SEQ. ID. NO. 8)

Cctra 893−: 5′ TCCATGATGTCGATATTGTCC 3′ (SEQ. ID. NO. 9)

Cctra 154+/T7: 5′ TAATACGACTCACTATAGGGCAGTGGTTCGGTTCGGAAG 3′ (SEQ. ID. NO. 10)

Cctra 893−/T7: 5′ TAATACGACTCACTATAGGGTCCATGATGTCGATATTGTCC 3′ (SEQ. ID. NO. 11)

The pair T7/154+, 893− has produced the substrate for the “sense” ssRNA (single strand RNA); while the pair 154+, T7/893− has produced the DNA template to obtain the “antisense” ssRNA. The amplification reaction has been preceded by a first step of denaturation of the template at 94° C. for 5′ and it has been performed as 35 reaction cycles, each of: 94° C. for 1′, 60° C. for 1′; 72° C. for 1′. The template used in this PCR is a fragment of the F1 cDNA (from the position 154 to 893). All the PCR steps has been conducted using DEPC treated water. Subsequently, the PCR products have been used directly for the in vitro transcription as suggested by the Kit protocol “in vitro RNA Trascription Kit” (STRATAGENE). Equimolar quantities of the sense and antisense ssRNA have been mixed into an annealing buffer (1 mM Tris pH 7.5; 1 mM EDTA) for 24 h to obtain dsRNA (double stranded RNA). The dsRNA has been transferred into an injection buffer (5 mM KCI; 0.1 mM NaPO₄ pH 7,8) at a final concentration of 5 mM. The injection technique of the dsRNA is essentially similar to the one invented to transform the Drosophila melanogaster germline (Rubin and Spradling, 1982) with the adaptations described by Kennerdel and Carthew (1998). The embryos were collected every hour, then manually dechorionated, by insulin syringe of 1 ml, dryed for 60-120″, covered by Halo-carbon oil 700 (Sigma) and injected, a group into the anterior pole and another group into the posterior pole.

### Example 6

Maintenance of the Benakeion and W/W

The Benakeion and white eye strains, used to obtain the embryos to be injected, have been reared as follow. The adult flies have been reared into two separated cages (40 cm×40 cm×32 cm), built up in a way to obtain the deposition of eggs into water containing boxes. The water contained into these boxes is poured on to a net to collect the embryos and to transfer them into Petri dishes containing larvae food made of a 400 ml whip of H₂O, 30 g of towel paper, 30 g of yeast extract (Laboratorio Dott. Piccioni, Gessate, Milano), 10 ml of “stock colesterol” (140 ml H₂O, 50 ml EtOH 95%, 10 g Cholesterol C₂₇H₄₆0, Sigma), 8.5 ml of “stock benzoic acid” (50 g Benzoic acid C₇H₆O₂, 300 ml EtOH 95% and 150 ml H₂O) 10 ml of “HCl stock” (384 ml H₂O and 66 ml HCl 37%). The Petri dishes in which the embryos have developed into larvae are then transferred into closed boxes containing some sand. The boxes are closed because the larvae, by a body contraction, jump into the sand (which is useful to avoid that the larvae stick together), where they will become pupae. The pupae are then separated by a sieve which let the sand pass and then they are transferred into Petri dishes. After about 10-13 days adult flies that come out of the pupae are transferred into the cages previously described (all the cycle lasts about 1 month at 25° C.). The maintenance of the dsRNA injected flies is similar to the one previously described. Indeed the injected embryos have to been transferred, when they develop into larvae, from the mineral oil into the Petri dishes containing the larvae food. This step is carried out under the stereomicroscope with the help of 1 ml insulin syringe. The remaining part of the vital cycle follows a normal development. The adult flies are then analyzed under the microscope, photographed and possibly frozen at −80° C. to be used for subsequent molecular analysis.

References

Anleitner, J. E. and Haymer, D. S. (1991). Y enriched and Y specific DNA sequences from the genome of the Mediterranean fruitfly, Ceratitis capitata. Chromosoma 101, 271-278.

Beverley, S. M., and Wilson, A. C. (1984). Molecular evolution in Drosophila and higher diptera. II. A time scale for fly evolution. J. Mol. Evol. 21, 1-13.

Boller, E. F. (1987). Genetic control in integrated pest management (ed. A. Berns, T. Cocker & P. Jepson), Academic Press LTD. 161-187.

Burtis, K. C. and Baker, B. S. (1989). Drosophila doublesex gene controls somatic sexual differentiation by producing alternatively spliced mRNAs encoding related sex-specific polypeptides. Cell 56, 997-1010.

Carroll, S. B. (1998). From pattern to gene, from gene to pattern. Int J Dev Biol.;42(3):305-9.

Cline, T. W. (1993). The Drosophila sex determination signal: how do flies count to two? Trends in Genetics 9, 385-390.

Christenson, L. D., and R. H. Foote. (1960). Biology of fruit flies. Ann. Rev. Ent. 5:171-92.

Fire, A., Xu, S., Montgomery, M. K., Kostas, S. A., Driver, S. E., Mello, C. C. (1998). Potent and specific genetic interference by double-stranded RNA in Caenorhabditis elegans. Nature 391(6669), 806-811.

Fisher, K. (2000). Genetic sexing strains of mediterranean fruit fly (Diptera: Tephritidae): quality in mass-reared temperature-sensitive lethal strains treated at high temperatures. J. Econ. Entomol. 93(2):394-402.

Fortier, E. and Belote, J. M. (2000). Temperature-dependent gene silencing by an expressed inverted repeat in Drosophila; Genesis 26, 240-244.

Furia, M., Artiaco, D., Saccone, G., Vito, P., Peluso, I., Giordano, E., and Polito, L. C. (1993). “Search for sex-specific genes in the medfly Ceratitis capitata: preliminary data on Sxl”, Management of Insects Pests: Nuclear and Related Molecular and Genetic Techniques (Proc. Symp. Vienna 1992), IAEA Vienna 215-224.

Gibbs, R. and Eerde, E. (1981). Irradiation proving successful in eliminating the medfly. R&D Mexico. 1: 16-18.

Goto, A., Matsushima, Y., Kadowaki, T., Kitagawa, Y. (2001). Drosophila mitochondrial transcription factor A (d-TFAM) is dispensable for the transcription of mitochondrial DNA in Kc167 cells. Biochem. J., 354 (Pt 2):243-248.

Irminger-Finger I. and Nothiger R. (1995). The Drosophila melanogaster gene I(3)73Ah encodes a ring finger protein homologous to oncoproteins MEL-18 and BMI-1. Gene 163(2), 203-208.

Kennerdel, J. R. and Carthew, R. W. (1998). Use of dsRNA-mediated genetic interference to demonstrate thet frizzled and frizzled 2 act in the wingless pathway. Cell 95,1017-1026.

Kennerdell JR and Carthew RW (2000). Heritable gene silencing in Drosophila using double-stranded RNA. Nat. Biotechnol.w 18(8):896-8.

Keyes, L. N., Cline, T. W., and Schedl, P. (1992). The primary sex determination signal of Drosophila acts at level of transcription. Cell 68, 933-943.

Knipling, E. F. 1955. Possibilities of insect control or eradication through the use of sexually sterile males. J. Econ. Entomol.48:459-62.

Yang, D., Lu, H., Erickson, J.W. (2000). Evidence that processed small dsRNAs may mediate sequence-specific mRNA degradation during RNAi in Drosophila embryos. Curr. Biol. 5;10(19):1191-200.

Lam, G. and Thummel, C. S. (2000). Inducible expression of double-stranded RNA directs specific genetic interference in Drosophila. Curr Biol 10(16):957-63.

Loukeris, T. G., Livadaras, I., Arcà, B., Zabalou, S. and Savakis, C. (1995). Gene transfer into the Medfly, Ceratitis capitata with a Drosophila hydei transposable element. Science 270, 2002-2005.

Louis, C., Savakis, C., Kafatos, F. C., (1987). Possibilities for genetic engineering in insect of economic interest. In: Fruitflies, Proc. II Intern. Symp., Economopoulos A. P., Ed. (Elsevier, Amsterdam), 47

Malavasi, A., Rohwer, G. G., and Campbell, D. S. 1994. In “Fruit Flies and the Sterile Insect Technique” Eds C. O. Calkins, W. Klassen and P. Liedo. CRC Press. 5 165.

Meise, M., Hilfiker-Kleiner, D., Dubendorfer, A., Brunner C., Nothiger R., Bopp D. (1998). Sex-lethal, the master sex-determining gene in Drosophila, is not sex-specifically regulated in Musca domestica. Development 125, 1487-1494.

McKeown, M., Belote, J. .,M, Baker, B. S. (1987). A molecular analysis of transformer, a gene in Drosophila melanogaster that controls female sexual differentiation. Cell. 13;48(3):489-99.

McKeown, M., Belote, J. M., and Boggs, R. T. (1988). Ectopic expression of the female transformer gene product leads to female differentiation of chromosomally male Drosophila. Cell 53, 887-895.

Meise, M., Hilfiker-Kleiner, D., Dubendorfer, A., Brunner C., Nothiger R., Bopp D. (1998) Sex-lethal, the master sex-determining gene in Drosophila, is not sex-specifically regulated in Musca domestica. Development 125, 1487-1494.

Mitchel, W. C. and Saul, S. J. (1990). Current control methods for the Mediterranean fruitfly, Ceratitis capitata, and their application in the USA. Review of the Agricultural Entomology. 78 (9), 924-940.

Misquitta, L. and Paterson, B. M. (1999). Targeted disruption of gene function in Drosophila by RNA interference (RNA-i): a role for nautilus in embryonic somatic muscle formation. PNAS 16, 1451-1456.

O'Neil, M. T. and Belote, J. M. (1992). Interspecific comparison of the transformer gene of Drosophila melanogaster reveals an unusually high degree of evolutionary divergence. Genetics 131, 113-128.

Polito, C., Pannuti, A., and Lucchesi, J. C. (1990). Dosage compensation in Drosophila melanogaster male and female embryos generated by segregation distortion of the sex chromosomes. Dev Genet. 1990 11 (4):249-53.

Rendon, P., McInnis, D., Lance, D., and Stewart, J. (2000). Comparison of Medfly male-only releases in large scale fild trials. In Area-Wide Control of Fruit Flies and Other Insect Pests, pag. 517-525, ed. K. H. Tan, Penerbilt Universiti Sains Malaysia, Penang.

Robinson, A. S., and Hooper, G. (1989). “Fruit Flies: their biology, natural enemies and control” Vol. 3A, Elsevier, Amsterdam.

Rong, Y., and Golic, K. C. (2000). Gene targeting by homologous recombination in Drosophila. Science 288 2013-2018.

Sambrook, J., Fritsh, E. F. and Maniatis, T. (1989). Molecular Cloning. A laboratory manual. II ed. Cold Spring Harbor Laboratory Press. New York.

Saccone, G., Peluso, I., Di Paola, F., Louis C. and Polito, L.C. (1996). Drosophila Sex-lethal and doublesex homologous genes in Ceratitis capitata: searching for sex-specific genes to develop a medfly transgenic sexing strain. Report First Research Co-ordination Meeting on “Enhancement of the Sterile Insect Technique through Genetic Transformation using Nuclear Techniques”, Joint FAO/IAEA, Vienna 30/9-4/10/96, 1-12.

Saccone, G. (1997). “L'omologo del gene doublesex di Drosophila melanogaster in Ceratitis capitata: evidenze di una parziale conservazione evolutiva nei due ditteri di una gerarchia di regolazione genica del differenziamento sessuale”. Tesi di Dottorato in Sistematica Molecolare, Relatore Prof. Catello Polito. Università degli studi di Napoli, “Federico II”.

Saccone, G., Peluso, I., Artiaco, D., Giordano, E., Bopp, D. and Polito, L. C. (1998). The Ceratitis capitata homologue of the Drosophila sex-determining gene Sex-lethal is structurally conserved but not sex-specifically regulated. Development 125,1495-1500.

Saccone, G., Pane, A., Testa, G., Santoro, M., De Martino, G., Di Paola, F., Louis, C. and Polito, L. C. (2000). Sex determination in Medfly: A Molecular Approach. In Area-Wide Control of Fruit Flies and Other Insect Pests, pag. 491-496, ed. K. H. Tan, Penerbilt Universiti Sains Malaysia, Penang.

Schutt, and Nothiger. (2000). Structure, function and evolution of sex-determining systems in Dipteran insects. Development 127, 667-677.

Shearman D. C. and Frommer M. (1998). The Bactrocera tryoni homologue of the Drosophila melanogaster sex-determination gene doublesex. Insect Molecular Biology 7,355-366.

Sievert, V., Kuhn, S., & Traut, W. (1997). Expression of the sex determining cascade genes Sex-lethal and doublesex in the phorid fly Megaselia scalaris. Genome, 40.

Sorensen., T. U., Gram, G. J., Nielsen, S. D., Hansen, J. E. (1999). Safe sorting of GFP-transduced live cells for subsequent culture using a modified FACS vantage. Cytometry 1;37(4):284-90.

Sturtevant, A. H. (1945). A gene in Drosophila melanogaster that transforms females into males. Genetics 30, 297-299.

Willhoeft, U. and Franz, G. (1996). Identification of the sex-determining region of the Ceratitis capitata Y chromosome by deletion mapping. Genetics 144, 737-45.

Zwiebel, L. J., Saccone, G., Zacharopoulou, A., Besansky, N. J., Favia, G., Collins, F. H., Louis, C., Kafatos, F. C. (1995). The white gene of Ceratitis capitata: a phenotypic marker for germline transformation. Science 270, 2005-2008.

1. Ceratitis capitata gene Cctra encoding the protein CcTRA able to regulate the sexual phenotype of dipteran species, in particular of Ceratitis capitata.

2. Cctra cDNA isolated and derived from the male-specific mRNA.

3. Cctra cDNA isolated female-specific indicated as Seq. IDN 1.

4. Nucleotide sequence isolated corresponding to the female-specific cDNA indicated as Seq. IDN 1.

5. Nucleotide sequence according to claim 4 which encode a polypeptide or a peptide comprised into the Seq IDN: 2.

6. Polynucleotide or oligonucleotide isolated comprising portions of the nucleotide sequence indicated as Seq. IDN1.

7. Polynucleotide or oligonucleotide 60% homologous or more to the polynucleodotide or oligonucleotide according to claim 6.

8. Polynucleotide or oligonucleotide 80% homologous or more to the polynucleodotide or oligonucleotide according to claim 6.

9. Aminoacidic sequence isolated indicated as Seq. IDN 2.

10. Cctra nucleotide sequence encoding for the aminoacidic region corresponding to the box 1 of FIG. 3.

11. Cctra nucleotide sequence encoding for the aminoacidic region corresponding to the box 2 of FIG. 3.

12. Cctra nucleotide sequence encoding for the aminoacidic region corresponding to the box 3 of FIG. 3.

13. Cctra nucleotide sequence encoding for the aminoacidic region corresponding to the box 4 of FIG. 3.

14. Nucleotide sequence complementary to the sequences according to claims 10-13.

15. Polypeptide or peptide 40% homologous or more to the aminoacidic sequences according to claims 10-13.

16. Polypeptide or peptide 60% homologous or more to the aminoacidic sequences according to claims 10-13.

17. Polypeptide or peptide 80% homologous or more to the aminoacidic sequences according to claims 10-13.

18. Antisense oligonucleotide able to hybridize with the mRNA derived from the cDNA according to claims 2 and 3.

19. Polynucleotide or polypeptide sequence according to previous claims to be used to regulate the sex determination of the dipteran.

20. Sequence according to claim 19 wherein the dipteran is selected among:
Glossina, Anophelese gambiae, Aedis Egypti, Ceratitis capitata.

21. Antibody against the Cctra protein.

22. Plasmid comprising the polynucleotide or oligonucleotide according to claims 2-8 and 10-13.

23. Plasmid according to claim 21 to be used as cloning and expression vector.

24. Vector including the polynucleotide and oligonucleotide according to claims 2-8 and 10-13.

25. Vector according to claim 23 further comprising a gene encoding a fluorescent protein.

26. Cell modified with a plasmid according to claims 21-22.

27. Cell modified according to claim 25 able to express the CcTRA protein or subfragments or its homologous proteins with a 40% of homology or more.

28. Pluricellular organism, exclude humans, comprising cells modified according to claims 25-26.

29. Transgenic non human animals “knock our” in which the Cctra gene is partially or fully inactivated.

30. Use of the nucleotide or polypeptide sequences according to claims 2-8 and 10-13, or parts of these sequences, as regulators of the sex determination in dipteran.

31. Use of the nucleotide or polypeptide sequences according to claims 2-8 and 10-13, or parts of these sequences, as regulators of sex determination in a dipterian selected among: Glossina, Anophelese gambiae, Aedis Egypti, Ceratitis capitata.

32. Method of gene silencing mediated by dsRNA molecules with a sequence specific for the Cctra gene according to the claim 1.

33. Method according to claim 31 performed injecting dsRNA molecules, artificially produced from a cDNA clone of Cctra into dipteran embryos to induce a reversion of the sexual phenotype from female to male one.

34. Method according to claim 32 in which the embryos are of Ceratitis capitata.

35. Method according to claim 32 in which the region chosen for the silencing is extended from the position 154 (exon 1) to the position 893 (exon 5) of the female-specific cDNA SEQ IDN 1 of Cctra and corresponds to exonic regions present into the clone of F1 cDNA-and into the M1 cDNA clone.

36. Method of gene silencing according to claims 31-34 performed by the use of a transformation vector by which an inducible transgene that produces Cctra—specific dsRNA molecules is introduced into the Ceratitis genome.

37. Method of gene silencing according to claims 32-35 in which antisense DNA or RNA molecules having specific sequences complementary to Cctra transcripts are injected or antibodies able to recognize the protein CcTRA^{F}.

38. Method according to claim 32 in which the injection is substituted with techniques selected between lipofection and electroporation.

39. Cctra gene according to claim 1 comprising 5 exons of which 2 are male-specific.

40. Process to produce Cctra dsRNA characterized by the use of 4 synthetic oligonucleotides named: 154+, 154+/T7, 893− and 893−/T7 with the sequence of the Cctra cDNA F1 as template.

41. Process according to claim 39 in which the oligonucleotides 154+/T7 and 893−/T7 show at the 5′ end the sequence of the promoter for the phage T7 RNA Polymerase.

42. dsR4NA produced according to claims 39-40 to be injected into the embryos of dipteran species at the posterior or anterior poles to obtain a sex-specific selection.

43. Use of the nucleotide sequence of the F1 cDNA containing the exonic regions of Cctra gene to eliminate from the progeny of the dipteran species, in particular of Ceratitis, the presence of the females transforming them into males.

44. Transgenic strain which produces by an inducible way dsRNA with sequences specific to the Cctra gene.

45. Cctra-GFP chimeric transgene encoding for a fluorescent protein.

46. Process to separate XX embryos from XY embryos characterized by the use of the transgene according to claim 44 which express sex-specifically fluorescent proteins of different colors.

47. Process to amplify Y-specific sequences by the Taq Polymerase characterized by the use of an oligonucleotide as primer selected among:
1-Y-SPECIFIC: 5′-GCGTTTAAATATACAAATGTGTG-3′ (SEQ. ID. NO. 3) 1 KbY-SPECIFIC: 5′-TACGCTACGAATAACGAATTGG 3′ (SEQ. ID. NO. 4)

48. Process to amplify a DNA fragment of the Cctra locus by the following sequence:
Cctra 1113-5′ CTGGAACTGGCACTGGTATTG 3′ (SEQ. ID. NO. 5)

49. Process to amplify an RNA fragment from males and females dipteran insects by PCR characterized by the use of the following primers:
F+=5′ CATGAACATGAATATTACAAAGGC 3′ (SEQ. ID. NO. 6)
Z1−=5′ CACGACGCTTATAGCTGTTGT 3′ (SEQ. ID. NO. 7)

50. Oligonucleotide selected among:
Cctra 154+: 5′ CAGTGGTTCGGTTCGGAAG 3′ (SEQ. ID. NO. 8)
Cctra 893−: 5′ TCCATGATGTCGATATTGTCC 3′ (SEQ. ID. NO. 9)
Cctra 154+/T7: 5′ TAATACGACTCACTATAGGGCAGTGGTTCGGTTCGGAAG 3′ (SEQ. ID. NO. 10)
Cctra 893−/T7: 5′ TAATACGACTCACTATAGGGTCCATGATGTCGATATTGTCC 3′ (SEQ. ID. NO. 11)
to obtain the dsRNA of Cctra.


SEQUENCE LISTING (11 sequences)

SEQ ID NO: 1 - DNA, 1584 bp, Ceratis Capitata
  CDS (89)..(1378): ccTRA cDNA
aattggtaaagaaaagcaacattaaacaacaggcaacgcatattgagttgaagaaacctatcaagtcacttcgttgacatctctcaacatgaacatgaatattacaaaggcttcggcaacaactaggaaaatacgaattgaacaaaatgtacccagtggttcggttcggaagggtccttacgctatagagcgcagtgtcaatccaagcgaagttgttattaaacgtagatttggtgaaggctcaaagcctctgtttcaacgcgacgacatcgttgtaaaccctgataatgttgtaagtaatgtgggtgctcattttgaaacacagcctaaagatcggagtaacaacagtaaagaggaggttgaaaatcaatggcgcaaagagcgtcacaaatcgacagattccagcagcccagaacgatttcgtaagcatcattctagtaacaaaagtgaacattcaaacagcggtaacaatattacaacacgacacacaaaaacgcaccacccttcacaagagaatcttaacacagcttcaaaacgccgtgactcttcaccaccaactaaccgacggcacagaacgccagagaaagttccctacttcatagacgagattagagaacgcgaccgcattcgacgtaaatatggaaagagatctacaaaatcaccgtcacccccagttatgtcatccaagtttcgtcgcaggcggagttactcaagatcaatctctcgttcaaggtctcattctcccgcaagaagtaaaaatcgtacacatgtttatggctcactttcacgacgctcgtcttcagtggatcgttacataggcggtggtaggaagcggcgtcgagaaaatttacgcaccgaaagagaccgggatggacaatatcgacatcatggacacagatcggaagaacaggaacgctcacgtcgtggtcgcagcccacgagcacgtaccagatctcgaacacgttcaagagaacgttcaaagcatgttcgtgcacgaaatgacgagcgtaataaaaatctccacggcaatcatgatgagttgacaaatgccgagctaaatcaaagaaatttaacgcaaccgcaaataataacaataccagtgccagttccagcggatttcttgaattatgcatattccacttggccaactcaaacacaatggtctcatccaatgacgccgcctccgcgatatggtgctccggcgtatcatatgccaaccattttacccgcaactgttatgccgccaatgcgacctgcgttaccgccttacggcttacctcctcagcctatgcgttacggtggacgtggattaagatttcctcagcaacatgggccacgtccgtggcgtcctaattttcgcccaaaaacacacaaatagtggctgataaatttagattatagctctaatcatatgcctagataatatctaagtagcatgttctcatgtattttcatatattaccacaacagctataagcgtcgtgaagtaaaaaagatgatagttttaatatttaatgttcttttatttacaaatatatatatatgtatctatatatatgaatagaacaataaaaaaaaaaaaaa

SEQ ID NO: 2 - protein, 429 aa, Ceratis Capitata
MNMNITKASATTRKIRIEQNVPSGSVRKGPYAIERSVNPSEVVIKRRFGEGSKPLFQRDDIVVNPDNVVSNVGAHFETQPKDRSNNSKEEVENQWRKERHKSTDSSSPERFRKHHSSNKSEHSNSGNNITTRHTKTHHPSQENLNTASKRRDSSPPTNRRHRTPEKVPYFIDEIRERDRIRRKYGKRSTKSPSPPVMSSKFRRRRSYSRSISRSRSHSPARSKNRTHVYGSLSRRSSSVDRYIGGGRKRRRENLRTERDRDGQYRHHGHRSEEQERSRRGRSPRARTRSRTRSRERSKHVRARNDERNKNLHGNHDELTNAELNQRNLTQPQIITIPVPVPADFLNYAYSTWPTQTQWSHPMTPPPRYGAPAYHMPTILPATVMPPMRPALPPYGLPPQPMRYGGRGLRFPQQHGPRPWRPNFRPKTHK

SEQ ID NO: 3 - DNA, 23 bp, artificial sequence
  oligonucleotide
gcgtttaaatatacaaatgtgtg

SEQ ID NO: 4 - DNA, 22 bp, Artificial sequence
  oligonucleotide
tacgctacgaataacgaattgg

SEQ ID NO: 5 - DNA, 21 bp, Artificial Sequence
  oligonucleotide
ctggaactggcactggtattg

SEQ ID NO: 6 - DNA, 24 bp, Artificial Sequence
  oligonucleotide
catgaacatgaatattacaaaggc

SEQ ID NO: 7 - DNA, 21 bp, Artificial Sequence
  oligonucleotide
cacgacgcttatagctgttgt

SEQ ID NO: 8 - DNA, 19 bp, Artificial Sequence
  oligonucleotide
cagtggttcggttcggaag

SEQ ID NO: 9 - DNA, 21 bp, Artificial Sequence
  oligonucleotide
tccatgatgtcgatattgtcc

SEQ ID NO: 10 - DNA, 39 bp, Artificial Sequence
  oligonucleotide
taatacgactcactatagggcagtggttcggttcggaag

SEQ ID NO: 11 - DNA, 41 bp, Artificial Sequence
  oligonucleotide
taatacgactcactatagggtccatgatgtcgatattgtcc